(19) United States
(12) Patent Application Publication
(10) Pub. No.: US 2024/0366238 A1
(43) Pub. Date: Nov. 7, 2024

(54) INSTRUMENT BOURNE POSITION SENSING SYSTEM FOR PRECISION 3D GUIDANCE AND METHODS OF SURGERY

(71) Applicant: Surgical Targeted Solutions Inc., Akron, OH (US)

(72) Inventors: Ian KAY, Fairlawn, OH (US); Quang-Viet NGUYEN, Aldie, VA (US); Rick FRANK, Newtonville, MA (US); David KAY, Akron, OH (US); Bryan DEN HARTOG, St. Paul, MN (US); Peter ONEILL, Bratenahl, OH (US)

(21) Appl. No.: 18/580,759

(22) PCT Filed: Jul. 14, 2022
(86) PCT No.: PCT/US2022/037128

§ 371 Date: Jan. 19, 2024

Related U.S. Application Data
(60) Provisional application No. 63/223,370, filed on Jul. 19, 2021.

(51) Int. Cl. A61B 17/16; A61B 34/00; A61B 34/30; A61B 90/00

(52) U.S. Cl. CPC A61B 17/1622; A61B 34/25; A61B 34/30; A61B 90/37; A61B 2034/305; A61B 2090/376

(57) ABSTRACT

A medical devices en-vivo positional determination system and method of surgery using a registration system to create a 3D frame of reference from 2D images and in which the user uses his or her judgement to select points of interest within the 3D frame of reference more accurate hand-held drilling or cutting, using forward kinematic equations calculated on a microcomputer to provide a real-time display of the 3-linear position and 2-angular orientation of a hand-held medical instrument which is linked by a draw wire or a virtual draw wire to a gimbal so as to measure the angle and distance of the instrument from a fixed point.

### CROSS-REFERENCE TO RELATED APPLICATIONS

This application is a national stage application, filed under 35 U.S.C. § 371, of International Patent Application No. PCT/US2022/037128 filed on Jul. 14, 2022, which claims the benefit of U.S. Provisional Application Ser. No. 63/223,370, filed Jul. 19, 2021, the entire disclosures of which are incorporated herein by reference for all purposes.

### TECHNICAL FIELD

The present disclosure relates to medical devices, and more specifically, medical devices used by qualified personnel such as physicians and nurse practitioners, and most notably surgeons of various specialties including orthopedic generalists, orthopedic and podiatric extremity specialists, spinal surgeons, neurosurgeons, oral surgeons, and dentists, during medical or dental procedures, and especially surgical procedures. More specifically, the disclosure provides a system that is related to relatively small and cost efficient hand-held surgical devices, such as a drill or wire driver, and tools or apparatus which can be sterilized, or which have a cost structure that would permit single use so that they are “disposable”, and to methods of surgery that incorporates such devices. Additionally, the system described herein permits fine precision control of an instrument so as to enable the user to manipulate the instrument in a reference system in 3D space aided by coordinated 2D images taken in differing planes, including fluoroscopic images so as to enable the guidance of the instrument to an internal point within the reference system but obscured from normal view because it is within the body of a patient. An operative planning method uses the system described herein to allow the normal use of a C-arm for diagnosis and patient specific 3-D planning and execution without the need for the cost of time for MRI or CT scans and analysis.

### BACKGROUND

While there has been a substantial body of work and commercial products which provide imaging assistance or robotic guidance, (i.e., “surgical navigation”) during surgery, the devices have been “large box” devices for example million-dollar devices owned and leased to the practitioner by a hospital or healthcare institution, and that are lodged in dedicated surgical environments. These devices require a very large capital investment, which includes the cost of the surgery room and environmental controls, training for dedicated personal, and an expensive and complex device. Moreover, these devices tend to be large and invasive in the surgery and may even dictate the surgical environment such as the space and temperature requirements around these devices.

Since these “big box” devices include complicated hardware and software and very high development costs, there has been relatively little development with respect to lower cost hand-held surgical devices with positional feedback, or “targeting systems”, for medical use since these devices have limited cost elasticity, and uncertain return on the development and production costs, in addition to cost absorption, payment or reimbursement issues.

Thus, typical “targeting” is presently limited to the hand-eye coordination of the practitioner performing the procedure. As discussed herein “targeting” refers to the guidance in time and through space of the trajectory and depth of an instrument workpiece within a biological environment, which typically involves highly sensitive areas and highly critical positioning and time constraints. Depending on the medical specialty or even the area of the body being treated, the “work path” may have constraints that include the start point, the end point, and the path between, especially for areas with high concentrations of sensitive and functional or life-threatening implications, such as the spine, extremities, the heart or the brain or areas critically close to nerves, arteries or veins. Thus, the system is intended for use in an area that has a volume ranging broadly from a cubic centimeter to a cubic meter with a radial end point accuracy of less than 3 millimeter, and preferably less than 2 or even 1.5 millimeters.

For procedures in which the precision of the cutting or drilling of a target pathway located within a physical patient body is crucial (i.e., the “work path”), the skill and hand-eye coordination of the surgeon is of paramount importance. Due to the nature of hand-held tools, and the dynamic and flexible nature of the “work area” within a patient body, errors of the tool tip versus ideal positioning during use can, and will, occur regardless of the skill of the working practitioner. This possibility is increased with user fatigue that can be physical and mental in origin, as well, as issues relating to inexperience, and differing surgical conditions, such as bone or soft tissue quality.

It is the aim of the present disclosure to reduce these errors by providing the surgeon with a real-time indication of the “work path” of the tool, based upon the sensed location of an attribute, such as a vector, of the instrument in space and coordinated to a point within the anatomical site (and behind and obscured by surrounding flesh and skin at the patient surface). Moreover, this point or “target” or “loci” can be defined using two-dimensional fluoroscopy images, preferably at least two taken at different planes, such as by using the now ubiquitous C-arm devices found in a typical surgical setting. Thus, an aspect of the disclosure relates to the registration and coordination of the instrument within a defined reference frame which includes the anatomical portion of the patient in question. The disclosure has the further goal of reducing the need for multiple fluoroscopic images particularly for minimally invasive procedures in which the inability to view the actual point of interest within the anatomy leaves the surgeon guessing what's inside based on 2D images and externally palpated bony landmarks. Moreover, the traditional use of 2D x-rays leaves the surgeon the task of coordinating two differing views so as to create a virtual 3D reference for the purpose of determining where a point of interest within the body. Under the best of circumstances, this translation is difficult, but it is even more troublesome under the time constraints and pressures of surgery. The present disclosure lets an expert view the 2D images and pin the point of interest in 2 views, and the software system then coordinates these two images to locate the 3D location of the point of interest in the reference system. This frees the surgeon from the burden of having to coordinate and remember the locate in 3D within the body.

A further aspect of the disclosure relates to the creation of a reference system which allows the location of points of interest of anatomical portion of the patient within that system. In particular, the system relies on the judgement of the surgeon during the operation to choose the points of interest, such as by setting a target or loci. These points are typically unseen and unseeable to the surgeon, except using an imaging technic that provides vision within the body. In the case where bones are involved, this means that the surgeon can choose a location within or through a bone, and the system can help to guide a procedure to that location. Alternatively, the system described herein can be used to operate within alternative body parts, including organs, and soft tissue.

In certain types of surgery, real-time radiography using x-rays provides the surgeon with the knowledge of positional information that would otherwise by invisible due to the opaqueness of the site. However, this is not always possible, and certainly, it is not desirable to use radiography in real-time as the exposure to x-rays can be considerable for both the patient and the surgeon. Thus, it is desired that the position of the tool tip relative to a desired “work path” be provided by a means that minimizes any health risk as a result of the surgery to the patient or surgeon.

While surgeons are presently accustomed to the use of C-arms as tools to “see” into the body, and this tool represents the current standard of care in operating room equipment, these devices are subject to the forces of gravity and movement in being re positioned during and more importantly, between surgical procedures. It is not uncommon, that they are “banged about” being moved from one surgical room to another. It has been recognized by the present inventors that this can significantly affect the internal calibration of the tool. Consequently, the present disclosure also relates to a method of compensation for possible distortion from the fluoroscopic device or the imaging system used with the present system described herein.

Additionally, it is important that any surgical aid include a method of use that results in a surgical workflow that is efficient and facilitates the procedure rather than obscuring it. Thus, the present disclosure further provides a method of use that optimizes the use of the present targeting or robotic system, and which enables an efficient intraoperative diagnosis and planning procedure. Thus, in the case of an elderly patient who falls from bed, the patient can go immediately to an operating room for a diagnostic x-ray which is also used for intra-operative planning, allowing the surgeon to stabilize a broken greater trochanter immediately, without the wait for an MRI or CT scan and analysis.

The present system described herein is also useful as a surgical simulator as a teaching aid to acquire the proper feel of the instrument through repetitive use in a replaceable bone sample, such as a saw bone, in a surgical setting and using a pre-arranged x-ray set-up and jig to hold the bone in a repeatable location.

### SUMMARY

The present disclosure addresses the need for a device which is distinguished from the prior art high capital “big box” systems costing hundreds of thousands of dollars and up. This disclosure further relates to a method for the accurate real-time positional determination in three dimensions of a surgical instrument workpiece relative to the end point or pathway within the patient body (i.e., the “optimal course” or “work path” of the instrument workpiece) in the operating room, for procedures including, among other things, drilling, cutting, boring, planning, sculpting, milling, debridement, where the accurate positioning of the tool workpiece during use minimizes errors by providing real time positional feedback information during surgery and, in particular, to the surgeon performing the procedure, including in an embodiment in line of sight, or in ways that are ergonomically, advantageous to the practitioner performing the procedure.

In a narrow recitation of the disclosure, it relates to a guidance aid for use by orthopedic surgeons and neurosurgeons that is attached to a standard bone drill or driver and operates so as to provide visual displayed feedback to the surgeon about how close the invasive pathway is during the drilling operation to an intended orientation and trajectory. Thus, the system described herein permits the surgeon to use the visual feedback to make course corrections to stay on track, and as necessary to correct the trajectory of a workpiece. In the past, surgeons would use a mechanical “jig” to help guide the position of the intended starting point, and the end point of a drill pathway (i.e., the drill hole), but the present system described herein uses electronic, and preferably optical time-of-flight (OTOF) sensors in collaboration with inertial measurement units (IMUs) and a digitally encoded extendable link or cable, the so-called “Draw-Wire” sensors, that are borne by a hand held instrument with a visual display and feed-back system to inform the surgeon as to how to create a drill pathway through a subject patient body part which is contained within a three dimensional reference frame. By “hand-held”, it is meant an instrument that weighs under five pounds and has a configuration that allows it to be manipulated in the hand of a user. Reference points are obtained such as through digital images, for example, captured using fluoroscopy.

The system provided herein uses an imaging system (which may be independent of the system or incorporated with the device) to establishes a frame of reference for the anatomical subject area to allow the system, including through the interaction of a user, to recognize and as necessary mark or “pin” reference points. The system provides for the placement of radio-opaque markers (e.g., multiple point fiducials in a known and recognizable geometric configuration) which are used to define related anatomical locations within the frame of reference and ultimately to allow a calibration of the absolute position of the hand-held sensor relative to the physical setting. Advantageously, the markers are provided in a spiraling geometry and within a radio-translucent block, that can be mounted from guide wires implanted in the anatomy of interest. This allows a simple and compact reference frame creation and for registration of the instrument within that reference frame.

The reference system that also includes the patient and a side plane, and an independent imaging system is used to visualize the anatomical site, while the system includes means to determine, and mark starting and end points, including using the judgement of the surgeon, relative to the anatomical subject area and input them into the reference system. The guidance system works within the marked reference area to determine the location of sensors, preferably OTOF, and kinematic IMU, and Draw-Wire sensors (although it should be understood that in certain aspects of the system other types of sensors and other types of imaging systems, can be used), carried on the hand held instrument which is linked by a flexible and extendible rod or cable to a base tied to the surgical site at a known relationship. Alternatively, in accordance with other aspects of the system, the instrument may be tethered to a virtual version of the draw wire, such as by using an optical tracking system or line of sight-based version or alternatively, using sound waves to accomplish the tracking of the instrument in the frame of reference.

Thus, the disclosure relates to a surgical targeting system guided by OTOF and kinematic sensors that are strategically mounted on the hand-held (or potentially robotic) drill. The sender receiver pairs are in proximity to x-ray opaque fiducials which are positioned relative to the subject surgical area (i.e., the anatomy of the patient which is located within a defined three-dimensional reference frame) and which determine the proximity in space of the associated OTOF and kinematic sensors as they change course over time. The markers and the drill entry and end points are selected by the user (surgeon), although it should be understood that they can also be selected using artificial intelligence or another machine based system, and entered into a computer program residing on a CPU member that accesses software to display or represent the drill pathway of the surgical workpiece in the subject surgical area on a GUI (“graphical user interface”) as determined by the relationship between the OTOF transceiver with the reference frame of the system. Thus, the system allows the display to inform the user as to the trajectory of the instrument and the depth of penetration into the anatomical site which can be displayed in a number of ways, including reticles or cross-hairs, circle in circle, numbers, colored lines showing the desired and actual course or vector, or other alignment methods including in separate visuals or combined.

In accordance with the present disclosure a plurality of OTOF (Optical Time of Flight) sensors acting as light pulse transceivers are mounted to the tool handle and relative to a reference frame that is represented by a base plate which is positionally fixed relative to the surgical site (i.e., the physical environment within or about the patient's body). In this case, the surgical site may also need to be positionally fixed or restrained within the reference frame. An electronic microprocessor system synthesizes the light pulses which are generated by the OTOF transceiver sensors, along with kinematic position and digitizes the measured received light pulses and performs the necessary algorithms such as FFTs (Fast Fourier Transform), correlation functions, and other digital signal processing (DSP) based algorithms performed in hardware/software, thus provides the real-time positional information for the surgeon for example, via an electronic screen such as in “line of sight” on the tool handle itself or on a separate monitor, including a display that could be linked to the system, such as on a head's up display screen worn by the surgeon or a dedicated display that is located at a position that is ergonomically advantageous for the user. The tool can be any tool used by a medical practitioner, including for example, a scalpel, saw, wire driver, drill, laser, arthroscope, among others.

In the simplest embodiment, the tool handle will support and/or house a plurality of the OTOF transceivers mounted in an orthogonal fashion along with an IMU and draw-wire sensor system such that 5 degree of freedom (DOF) information regarding the linear (x, y, z) position, and the angular (yaw, pitch) can be obtained from the knowledge of the vector positions. At a minimum there is 1 OTOF transceiver, an IMU, and a draw-wire sensor, but preferably 3 OTOF transceivers to provide redundancy.

By means of the targeting assistance provided by the present disclosure, it is further desired that 5 degrees of freedom (DOF) positional information be provided in real-time at rates of up to 3, preferably 2 and most preferably 1 per second, with a positional accuracy of +/−3 mm, preferably 2 mm, and most preferably 1 mm, in 2 or 3 linear dimensions, and angular accuracy of +/−3° and preferably 2 in 2 angular dimensions of pitch and yaw, and that this positional information be obtainable in a 0.75 m×0.75 m×0.75 m, and preferably 0.5 m×0.5 m×0.5 m cubic working volume.

In the present disclosure, a plurality of OTOF transceivers (i.e., at least 3 and more precisely from 3 to 15, or 3 to 10 where the excess from a three-dimensional matrix are used for an array) are used to provide the positional information of a tool relative to a mechanical reference plane supported or mounted relative to or on the tool. The distances from the transmitters to the transceivers are calculated either by a time-of-flight (TOF) propagation of the transmitted sound pulse, or based on the phase information from the Fast Fourier transform (FFT) of the light waves emitted from the transmitter(s) onto the receiver(s) on the OTOF sensor. This phase information is proportional to the time delay of the transmitted pulse to the received sound pulse. With the use of the speed of light, a distance from the OTOF transceiver can be calculated. Internally, to the OTOF sensor, the use of phase extraction from optical heterodyne techniques provides some immunity to amplitude noise as the carrier frequency is in the MFIz range and well above the usual 1/f noise sources. The use of certain coding schemes superimposed upon the carrier frequency permits the increase in signal to noise ratio (SNR) for increased immunity to ambient noise sources. Other means of extracting distance or positional information from ultrasonic transducers for robotic navigation have been described by Medina et al. [2013], where they teach that via use of a wireless radio frequency (RF), coupled with ultrasonic time-of-flight transducers, positional information with up to 2 mm accuracy can be obtained in a space as large as 6 m for tracking elder movement. Segers et al. [2014, 2015] has shown that ultrasonic pulses can be encoded with frequency hopping spread spectrum (FHSS), direct sequence spread spectrum, or frequency shift keying (FSK) to affect the determination of positions with accuracies of several centimeters within a 10 m space. More recently, Khyam et al. has shown that orthogonal chirp-based modulation of ultrasonic pulses can provide up to 5 mm accuracy in a 1 m space. Liao et al. showed that image guided surgery (IGS) could provide accuracies up to 2.5 mm. A more recent review of various IGS techniques shows a survey of prior-art techniques that combine image processing and radiography to enhance surgery outcomes via an improvement of the instrument placement accuracy. However, none of these previous studies have been able to provide a 2 or 1 mm accuracy for a system that fits within an operational size space that is the size of the intimate volume directed affected by most medical procedures (i.e., about 1 cubic meter or less), which is the goal of the present disclosure.

In a further embodiment, the tool and the base for the workpiece can also contain visual fiducial markers that will assist a double set of video cameras mounted orthogonally as to produce a top view and a side view so that the fiducial markers can be used with video image processing to deduce spatial information that can be used in conjunction with the OTOF sensors for positional information.

In yet a further advanced embodiment, the digital signal processing (DSP) and sensor fusion of the various data streams from the OTOF, IMU, and draw-wire sensors will provide a precision virtual reality high-dexterity effector to allow precision remote-controlled operations requiring great dexterity and control of a tool or instrument such as: surgery, bomb-defusing, spacecraft repair, etc.

In a third embodiment, the OTOF and kinematic sensor system above is used in conjunction with a fluoroscopic radiography system to provide both contextual imaging, coupled with quantitative positional information for the most critical types of surgery (which can include spinal surgery, invasive and non-invasive neuro-surgery or cardiac surgery, for example). Thus, the disclosure also relates to methods of performing medical procedures including surgery and dentistry that establishes a frame of reference for the anatomical site, and wherein a medical tool supports sensors to locate and guide a medical procedure on the anatomical site within the frame of reference. As an example, the present disclosure relates to a procedure involving a guided procedure to percutaneously implant guide wires in a femoral neck for a non-invasive cannulated screw fixation of a hip fracture.

All of the above embodiments allow for the real-time display of the absolute positional information of the tool workpiece and preferably the tool tip, relative to the body part, intended target position, and the desired “work path”. The display could show a delta distance reading relative to the intended target position so that the surgeon is simply looking to minimize the displayed delta numbers or a graphical or other visual representation thereof (e.g., circle in circle). Alternatively, the display can illustrate the instrument having a direction for a vector which it applies to the body and the vector can be aligned with a desired direction for the vector. The display will show the x, y, z positions to the nearest millimeter or partial millimeter and also the yaw and pitch to the nearest degree or partial degree, including the incremental changes of these values. The angle of approach is often an important parameter for certain procedures such as a wire drill and especially where the start point may be known, and the end point maybe marginally understood, but the path between may only have certain criteria.

It is also the aim of this disclosure to provide this positional information in a lightweight tool handle that is unobtrusive and easy to use, and as similar to the existing instrument as possible, such that the transition to use of the system of the disclosure is user friendly and seamless to the practitioner. It is a further goal of this disclosure to have a tool handle and base plate with transmitters that are easy to sterilize, including by autoclave, or which are cost-effective enough for manufacture in whole or in part, as a disposable one-time use system.

It is one advantage of the present system that it can be very compact and unobtrusive by nature of the form factor, and the possibility of being wireless, and the positional sensing is effected by light and a single absolute distance kinematic sensor compared to mechanical position sensors such as articulated multi-joint angular-feedback linkages, and further that the system can be safely used in a healthcare facility without hindrance by external noise or without contaminating other wave uses in the facility.

Another advantage of the present system is that it permits the surgeon to manually hold the tool in a natural manner that does not have any mechanical resistance, such as that might be encountered with as articulated multi-joint angular-feedback linkages, and with a footprint and size that can be easily manipulated and which is similar so much as possible to the tools that they are already comfortable using. This is particularly true in the embodiment in which the draw wire is a virtual draw wire.

It is another advantage of the present system that it can provide both position and angular information simultaneously, and advantageously, sufficiently in ‘real-time” to enable the use during surgery.

It is another advantage of the present system that it has immunity over typical ambient background noise sources since it works in the near infrared wavelength band, and the data processing occurs via FFT in the frequency domain where typical mechanical and ambient noise source amplitudes are minimized through the 1/f principle where noise amplitude is inversely proportional to the noise frequency.

It is another advantage of the present system that it can be used to augment radiography techniques such as fluoroscopy or x-rays to provide an additional level of information that is quantitative and can be used for the “last inch” deployment of a surgical tool for critical procedures where accuracy is of paramount importance.

It is another advantage of the present system that it provides the surgeon with positional sensing system that is absolute relative to the working base reference system and is free from dead-reckoning (propagation-based) errors that are inherent in some other types of (non-absolute) positional sensing.

It is an additional advantage of the system that it serves as a three dimensional aiming system based on present two dimensional imaging systems that a single use or low cost hand-held instrument includes a system that helps the user (a surgeon or robot) determine the work angle for a tool tip integral to the instrument from an identified point of entry in an anatomical work area to a desired end and provides haptic feedback by display or tactile means to correct the alignment of the tool tip to achieve and/or maintain the desired alignment. The system can be used in surgery, or for training purposes, with an instrument, such as a drill or wire driver or for the implantation of implants including pegs, nails and screws. Examples of suitable surgical methods using the present system include hip fracture fixation where a screw of nail is inserted into the greater trochanter using the present targeting, aiming or guidance system or instrument, or for use in hammer toe fixation which can include phalangeal intermedullary implants.

### BRIEF DESCRIPTION OF THE DRAWINGS

FIG. 1 shows a schematic diagram of the preferred embodiment of the present system;

FIG. 2 shows a schematic diagram of the principle of operation;

FIG. 3 shows a block diagram of the electronic system;

FIG. 4 shows a block diagram of the steps and sequence used to acquire and derive the distances and angles from the sensor data that are generated and collected;

FIG. 5 shows a photograph of a prototype of one embodiment of the present system reduced to practice;

FIG. 6 shows a photograph of the prototype of the present system with a detail showing the internal electronics in the base of the tool handle;

FIG. 7 shows a photograph of the system and held in a hand to demonstrate the ergonomic aspects;

FIG. 8 shows a 3-dimensional spiral fiducial reference system mounted on guide wires for registering the present system's coordinate axis system with the global coordinate axis system;

FIG. 9 shows a schematic representation of the 3-dimensional spiral fiducial reference system mounted on the guide wires with the present system and its use to register the spatial coordinate system of the X-ray C-arm and operating room coordinate system;

FIG. 10 shows a 3-dimensional perspective view of a C-arm X-ray machine with the present system and the associated local and global coordinate systems;

FIG. 11 shows a photograph of a first embodiment of the instrument in accordance with the system;

FIG. 12 shows an alternative embodiment of the instrument in accordance with the system having a draw wire sensor mounted on the instrument handle;

FIG. 13 shows a second alternative embodiment of the instrument in accordance with the present system which uses a wireless tracking device instead of the draw-wire of the present system;

FIG. 14 is a side bottom view of the registration phantom and mount guide pins from a first bottom side angle;

FIG. 15 is a side bottom view of the registration phantom and mount guide pins of FIG. 14 from a second bottom side angle;

FIG. 16 is a bottom side view of the fiducial block of the present system illustrating the geometric location of bores for locating the radio-opaque fiducials as well as registration points;

FIG. 17 is a side view of an x-ray showing the registration phantom and mount guide pins secured to a proximal end of a femur;

FIG. 18(a) is an illustration of an artificial femur positioned before a C-arm with the phantom mounted on guide wires implanted in the femur;

FIG. 18(b) is a fluoroscopic image of the femur and phantom shown in FIG. 18(a)

FIG. 19 illustrates the process of registering points from the 2D fluoroscopic images to create 3D coordinates in a 3D coordinate system for use in guiding an instrument;

FIG. 20 illustrates marking points of interest using the 2D fluoroscopic images to generate target points such as end points in the 3D coordinate system;

FIG. 21 illustrates the graphical user interface which guides the user in aligning the trajectory of the hand-held instrument in the 3D coordinate system;

FIG. 22 is a top side view of a distortion target assembly in accordance with the present system;

FIG. 23 is a top view of the distortion target assembly of FIG. 22 showing the calibration target;

FIG. 24 is a top side view of the distortion target assembly of FIG. 23 mounted to a C-arm;

FIG. 25 is a fluoroscopic image of the distortion target assembly of FIG. 22; and

FIG. 26 is a flow chart out-lining the surgical procedure in accordance with another aspect of the present system.

### DETAILED DESCRIPTION OF THE EMBODIMENTS

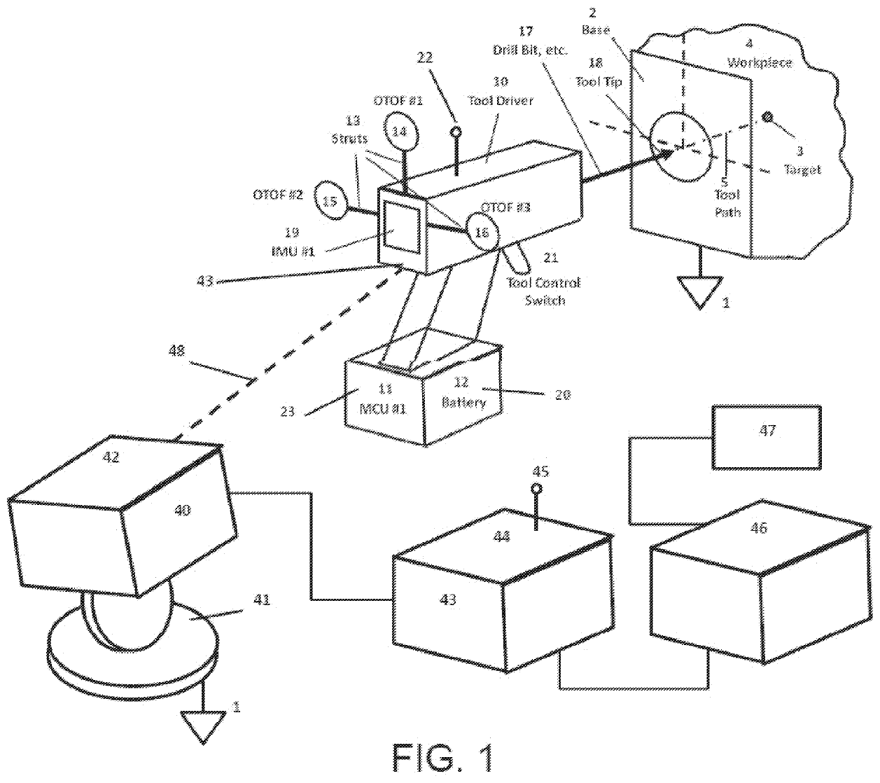

In the preferred embodiment of the present system as shown by the schematic diagram in FIG. 1, a tool driver 10, fitted with struts (supporting rods) 13 that serve to hold at least three OTOF transceiver at the top 14, left 15, and right 16 positions. The tool driver has a tool bit (k wire, drill, scalpel, etc.) 17, which has a distal tip 18 which corresponds to the spatial positional information shown in the display 47. The tool driver also has a k-wire/drill bit position sensor 17′ which provides a measurement of the extension of the drill bit relative to the tool handle. The drill bit position sensor 17′ uses a rotating wheel attached to a rotary shaft encoder that tracks the linear position of the drill bit as it is extended or retracted. The transceivers 14, 15, 16 (e.g., Sparkfun VL53LOX) are in optical communication with an optically reflecting flat base 2. These optical transceivers are optically linked to a rigid base plate 2 that serves to locate the transmitters with respect to the work path in the surgical environment in the patient's body part 4 subject to the procedure, to guide the tool tip 18 through an aperture 6 in the base 2, along the work path 5, towards the target 3. The OTOF transceivers, IMU 19, are in direct or indirect electrical communication with an electronic microcontroller unit #1 (MCU #1) 11 to a controller PC (or “CPU”, i.e., a computer processing unit), 46 via physical wiring cable or by radio frequency electronic transmission, such as Xbee or Bluetooth via RF transceivers 20 and 44 via antennas 22 and 45 and MCU #2 43. A draw-wire encoder 40 mounted on a rotating 2-axis gimbal mount 41 and physically linked through a flexible and extensible link, such as a mechanical tape, wire, rod, or most preferably cable 48 between the draw-wire encoder 40 and the tool handle 10, provides the absolute mechanical distance from a fixed reference mechanical ground point 1 to the target 3. The draw-wire encoder 40 also is fitted with an IMU #242 to provide the azimuth and elevation angles that are transmitted to the MCU #243 via wires and then to a PC controller 46 which performs calculations in software to fuse the data generated by the OTOF sensors, the two IMUs, and the draw-wire sensor into a real-time display of the positional information for the surgeon to use as feedback of the tool tip 18 position. The draw wire and gimbal which hold it serve as a flexible robotic arm, where the arm of the person holding the tool acts as a further robotic arm. In this regard, the present system uses the user to complete the robotic function and provides assistance to the user in determining the movement of the tool held in the arm. In a further embodiment the physical draw wire is replaced by a virtual draw-wire which is formed by an active motor controlled system in which a motor centers the point tilt angles of a camera in order to center a track sphere on the tracking system on the instrument. This changes the actual tether to a laser range finding device. Flere the base is orthogonal to the track sphere and TOF sensors are used for reflection back to the sensor system.

Together, these components shown in FIG. 1 form the basis of the tracking component of the present system's first embodiment that utilizes the measurement of the TOF (“Time of Flight”) of a light pulse from the transceivers 14, 15, and 16. By use of geometrical relationships, the fixed distances between the individual receivers and transmitters, and the speed of light, the angles of the OTOF relative to the draw-wire axis 42, the precise distances between the spatially separated transmitters and receivers can be determined with a closed form equation calculated either in the MCU #1 43, the computer 46, or even through use of a microcontroller MCU #1 11 in the tool driver 10 itself and then displayed on the screen 47. In this sense, the system can be predictive of the continued course of the tool-tip along the work path, although, it should be understood that the system tracks the position and displays it in near-real time during use.

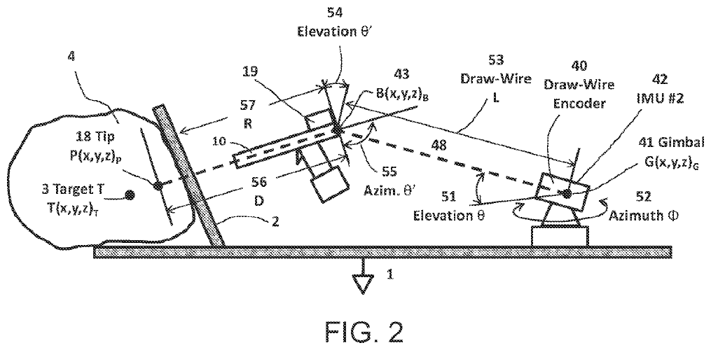

FIG. 2 schematically illustrates the principle of operation of the present system. Here, the drill handle 10 along with drill shaft 17, draw-wire sensor 40, IMU #1 19 and IMU #242 form a completely deterministic 2-link mechanical linkage system described by the so-called forward kinematic equations that are used for traditional serial link robotic arm analysis. Here, in FIG. 2, the arms have rotating joints located at 43 and 41 are free to move in elevation θ51 and azimuth Φ52 at the gimbal joint 41 and in elevation θ′54 and azimuth Φ′55 at the ball-joint attachment point 43. The elevation and azimuthal angles are provided by the IMU's 19 and 42 which are fitted with micro-electro-mechanical systems (MEMS) gyroscopes, accelerometers and magnetometers to effect angular measurements with 0.02 deg accuracy and essentially zero angular drift. In FIG. 2, the knowledge of the variable length L of the draw-wire 53, plus the distance from the drill tip 18, to the ball joint 43, plus the elevation and azimuthal angles at each joint as described above, completely describes the position of the tip 18, relative to the target T point 3 at (x,y,z)_{T}, and its trajectory as described by a vector transecting the points B at (x,y,z)_{B} and T at (x,y,z)_{T}. The position of any point in a serial chain of links can be described a transformation matrix as described by the so-called Denavit-Hartenberg parameters described elsewhere by Hartenberg and Denavit (1964). The OTOF distance sensors mounted on the drill handle are located at a distance R 57 from a reference plane 2 that is mechanically fixed to the patient 4 with target T 3, with the-patient 4, the reference plane 2, the gimbal 41 are all mechanically grounded to the reference frame 1. In this way, the relative position of the drill tip P 18 located at (x,y,z)_{P} and the target T 3 located at (x,y,z)_{T} relative to the gimbal origin point G 41 located at (x,y,z)_{G} are always known via the forward kinematic equations plus the absolute distance from the point B (x,y,z)_{B} to the reference plane 2 are also know to permit a redundant measurement of distance for error checking. Note that through the use of three OTOF sensors, the angle of the drill vector 17 relative to the reference plane 2 is also known and this provides a redundant measurement of the angle of approach as measured from the IMU sensors.

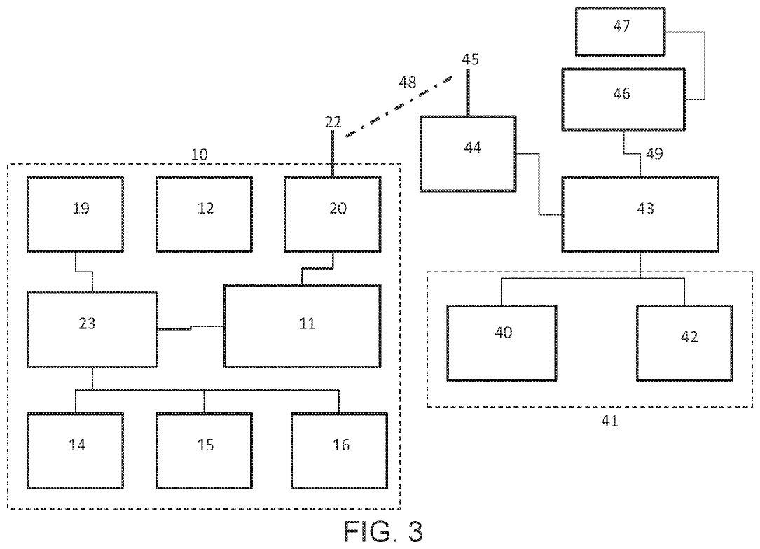

FIG. 3 shows a schematic block diagram of the electronics and their interconnections for the present system. The tool driver 10 shown by the dashed box contains the following electronic components which when connected, provide a measurement of the distances from the OTOF sensors 14, 15, 16 which are multiplexed through a MUX 23, and the angular orientation data provided by the IMU #1 19, and drill bit positional sensor 17′, which are all fed to a MCU #1 11 connected to a wireless RF transceiver link #1 20 fitted with an antenna #1 22. All components in the tool driver 10 are powered by a battery 12.

The battery can be rechargeable or of the primary type. The antenna 22 transmits the data in the drill handle 10 via an RF link 48, to a second RF link #244 also fitted with an antenna #245. The RF link #245 then sends the wireless data from the tool driver 10 to a second MCU #2 43 which also collects data from draw-wire base 41 which contains the draw-wire encoder 40, and the IMU #242, and all these data are then processed and fused together via a software program (such as MATLAB or Python) in a PC computer 45 via a USB link 49. It is also possible to replace MCU #243 with a more powerful MCU or a single board computer (SBC) to affect the calculations performed in the PC 46. The final positional information and angular data are then presented to the operator via display screen 47.

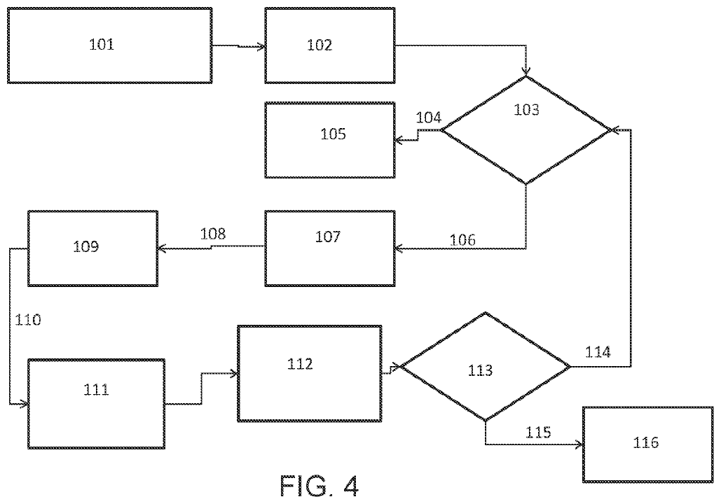

FIG. 4 shows a block diagram of the top level software steps used to calculate and derive the spatial measurement using the system depicted in FIG. 1. In the first Step 101, the MATLAB program initializes the serial communications interfaces between all of the interconnected devices, and in Step 102, the MCU's accepts an identification number and starts the program. In Step 103, the MATLAB program sends a Mode 1 or Mode 2 depending on whether or not the program is starting and being initialized. In the case of a start of initialization, Mode 1 is selected which then initialized all of the arrays in the MCU's in Step 105. Once that is done, Mode 2 is selected by the MATLAB program in Step 106 and the MCU's record the orientation and raw distance data from the sensors, whereupon the MCU sends the parameters to the MATLAB program via a serial link in Step 108. In Step 109, the MATLAB program stores the values in a matrix, and these are used in the matrix transformation in Step 110 as described by the so-called forward kinematic equations. The MATLAB program then plots the link lengths and trajectories in a graphical user interface (GUI) in Step 111, whereupon the distances and angle inputs are then graphed on the GUI in Step 112. Depending on whether more data is needed or the sensors need to be stopped in Step 113, the MATLAB program sends a Mode 2 Step 114 to continue the measurement cycle or a Mode 3 in Step 115 to shutdown and stop the program execution in Step 116.

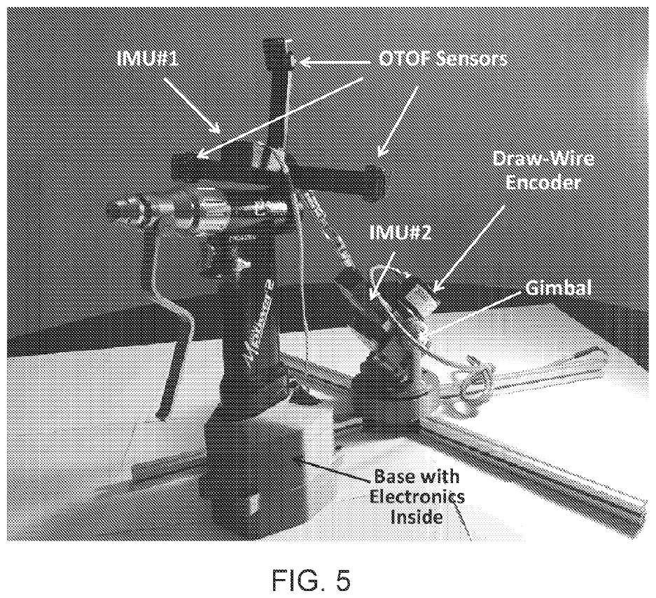

FIG. 5 shows the prototype of the present system. In FIG. 5, there are notations showing the locations of the OTOF sensors, the IMU #1, IMU #2, the draw-wire encoder, the gimbal base, and the drill handle base with electronics mounted inside.

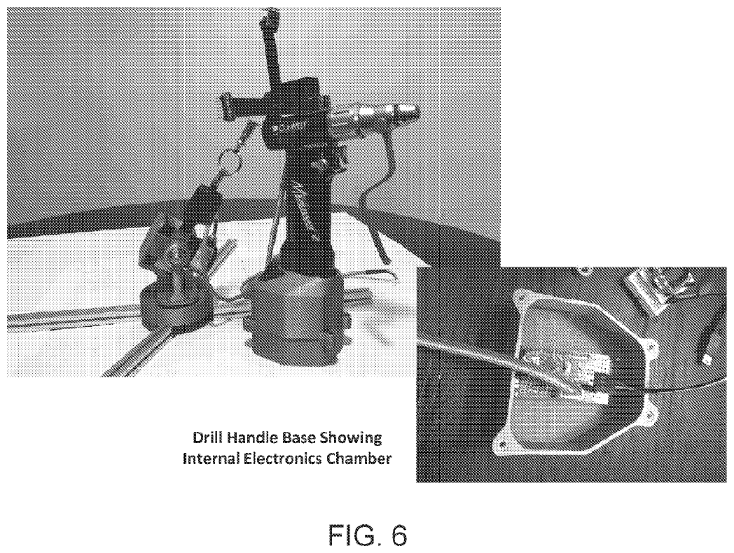

FIG. 6 shows another a different view of the present system from a different perspective for better clarity. Of note is a detail of the drill handle base with the cover removed to show the MCU #1 inside.

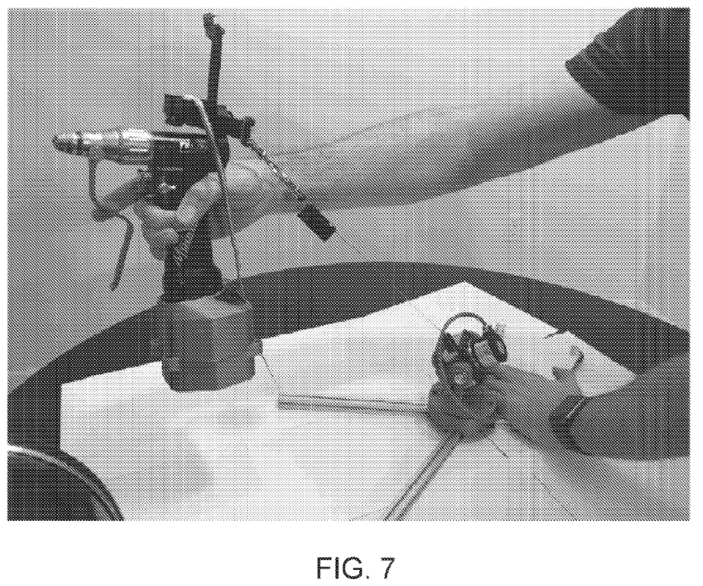

FIG. 7 shows the present system but being hand-held to show the relative positioning of an example of where the draw-wire encoder is located and how the gimbal mount allows the draw-wire orientation to be determined with an IMU #2 mounted in the gimbal head.

Analysis of the theoretical best accuracy of the positional determination using a first order angular resolution and moment-arm approach with the measured standard deviations from the IMU angular sensors (+/−0.02 deg) and variable length link arm from the draw-wire sensor (+/−0.5 mm), yields an approximate overall positional uncertainty in radial distances (x,y) of the drill tip to be +/−0.33 mm and axial distance (z) of the drill tip to be +/−0.71 mm. The present prototype embodiment is illustrated having relatively low-tolerance, non-rigid 3d printed plastic mounts used for the mechanical linkages, however, these will be replaced with precision low-backlash machined metal joints, to improve accuracy and to tend towards the theoretical limits shown above.

Analysis of the angular uncertainties of the IMU sensors yields and approximate angular uncertainty of +/−0.03 degrees in elevation (pitch) and azimuth (yaw).

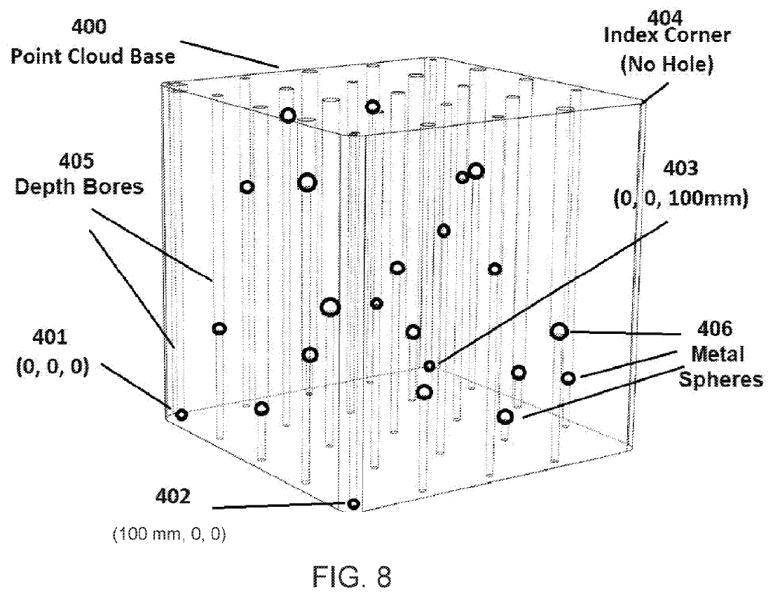

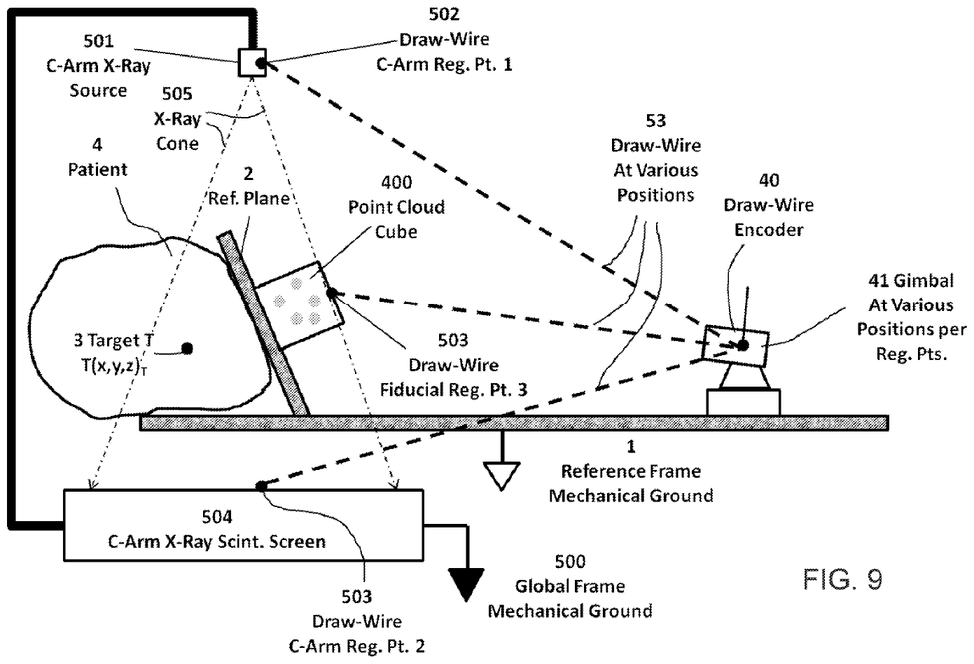

FIG. 8 shows a 3-dimensional geometrically defined array of radio opaque fiducials which are mounted or suspended in radio translucent block, that together form a point-cloud fiducial base or fiducial phantom 400. For example the block can have, for example, the shape of a cuboid or cylinder with precision-depth bored, cast-in, or machined holes 405 which support a plurality of metal spheres 406 of various diameters. At minimum, 3 spheres are required, with typically 8 to 12 spheres being desirable for the accurate calculation of the coordinate system position and orientation via a plurality of orthogonal X-ray images. The sphere positions are strategically chosen as pseudo-random (x, y, z) coordinates in such a way that their X-ray projections at two orthogonal axis do not occlude each other. Three of the spheres, preferably of the smallest diameter circa 2 mm are located on the bottom face of the fiducial cube at the (0, 0, 0), (100 mm, 0, 0) and (0, 0, 100 mm) positions to establish a reference frame with which to register against a flat reference surface representing the global coordinate system frame. A global coordinate system or frame of reference is defined as the frame of reference of the operating room as connected to the earth's surface. The local coordinate system or frame of reference, or more simply, reference system, is defined as the coordinate system associated with just the mechanical base 1 of the present system. The metal spheres 406 have various diameters (e.g., 2 mm, 3 mm, 4 mm, 5 mm) to aid in the identification of the orientation relative to a known arrangement within the cube. The cube should have a visual indicator 404, such as one corner that is not bored as a visual index for the user to place with a known orientation. Each sphere inside the fiducial base is at a precisely known position and these position coordinates can be used with fluoroscopy using a C-Arm apparatus as shown in FIG. 9.

In FIG. 9, the fiducial base 400 is mounted on a pair of guide wires which are implanted into the patient anatomy. And completely within the field of view of the X-ray cone 505 produced by the C-arm X-ray source 501. The X-ray cone 505 transects the fiducial cube 400, the patient 4 with desired target point 3, and projects the X-ray image onto the C-arm X-ray scintillation screen 506. The C-arm is anchored to the operating room global frame of reference 500, while the gimbal 41 and reference plane 2 are anchored to a local mechanical reference frame 1. By using the draw-wire 53 and gimbal base 41 to touch various points 502, 503, and 504, the relative positions and orientation of the C-arm, the reference frame mechanical ground 1, global reference frame ground 500, and the patient 4, can all be registered and linked together in a single solid body coordinate system.

In a further embodiment of the system, an IMU or other sensor can be mounted from an implanted guide wire in order to track movement of a bone of interest in the event that it can't be totally fixed within the reference frame.

Note that a minimum of 3 registration touch points are needed at each location 502, 503, and 504 to uniquely establish the 3-dimensional position and orientation of that part. By rotating the C-arm source 501 and scintillation screen 506 together and capturing at minimum, two orthogonal projections, the positions of the fiducial spheres in the point cloud base 400, can be uniquely established via linear algebraic methods as described by Brost et al. (2009).

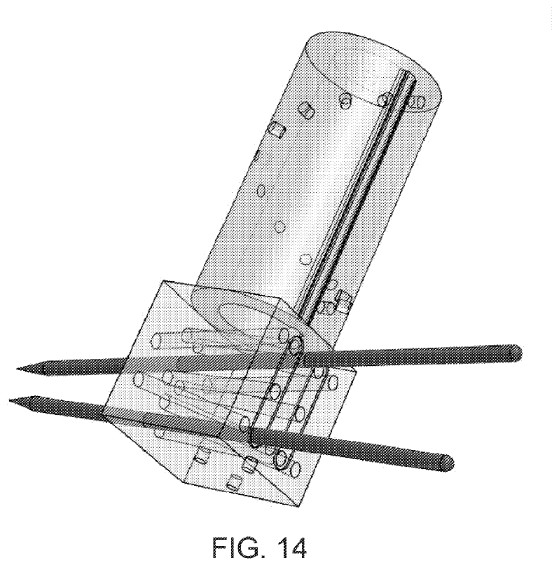

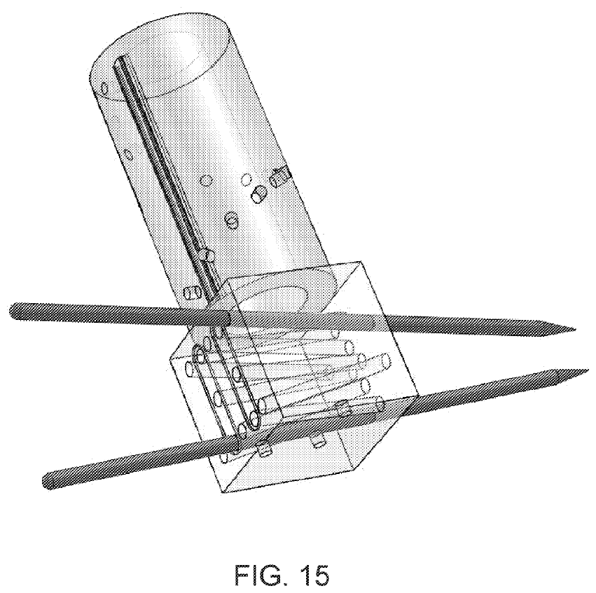

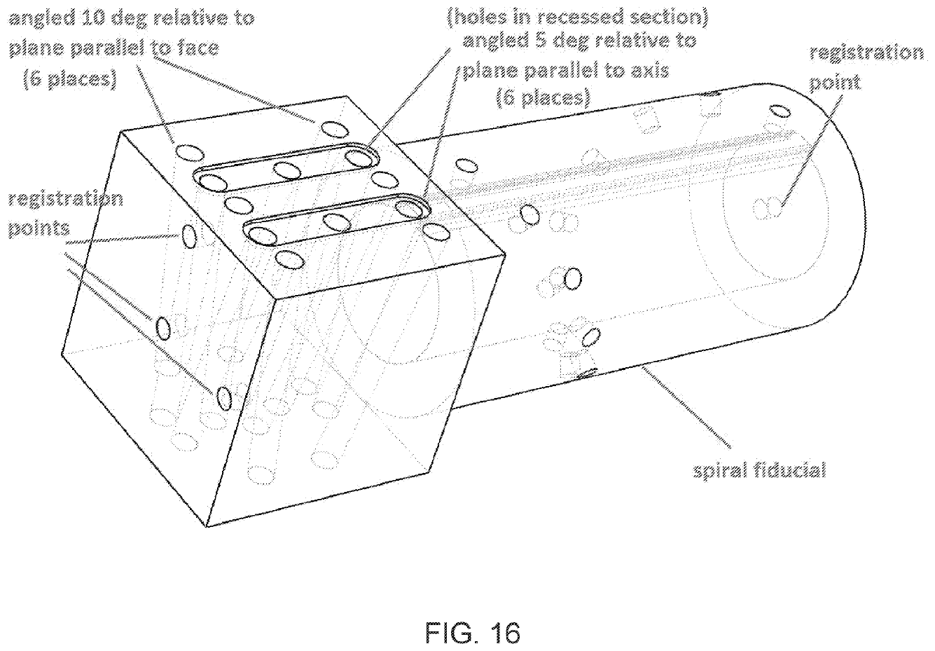

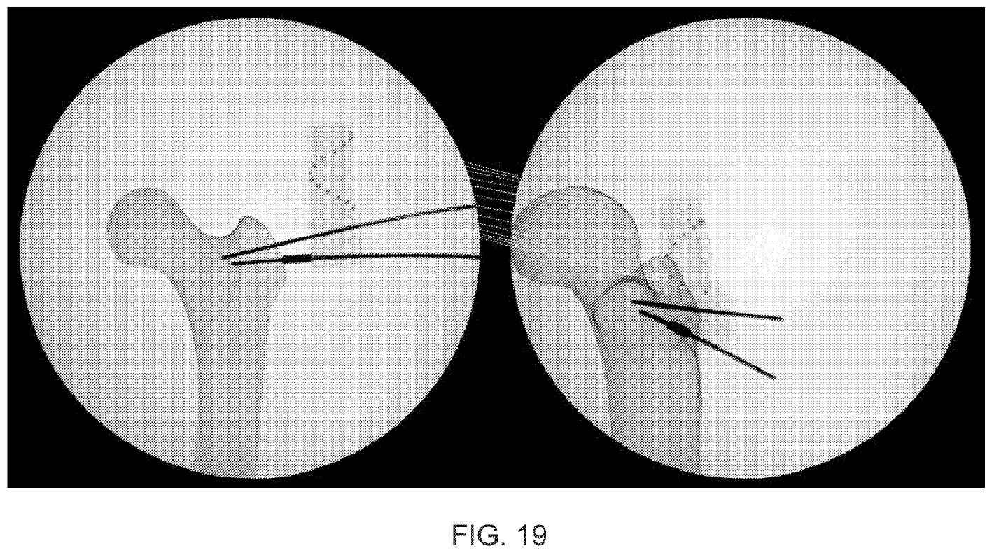

FIGS. 14-20 illustrate an additional embodiment of the fiducial base or phantom 1399 in which the block 1400 is cylinder with holes 1405 that are arranged in a known geometric array, in this case, a spiral which makes a full rotation about the length of the cylindrical block 1400. The phantom 1399 also includes a mount 1406 which includes through bores 1408. A first set 1412 of these bores are 1408 angled at from 5 to 15°+/−2.5° relative to a plane parallel to a reference plane of a reference coordinate plane of the system, while a second set 1414 are angled at from 2.5 to 7.5°+/−2.5° relative to a plane parallel to a reference plane of a reference coordinate plane of the system. The bores 1408 allow the phantom 1399 to be mounted from guide wire pins 1420, 1422 which are received in the bores to secure the phantom in a fixed position relative to the bone. One guide wire pin 1420 is held in one of the first set of bores 1412, and the second guide wire pin is held in one of the second set of bores 1414. The spiral shape of the fiducials allows the system to create a coordinate reference system in 3D from 2D x-rays.

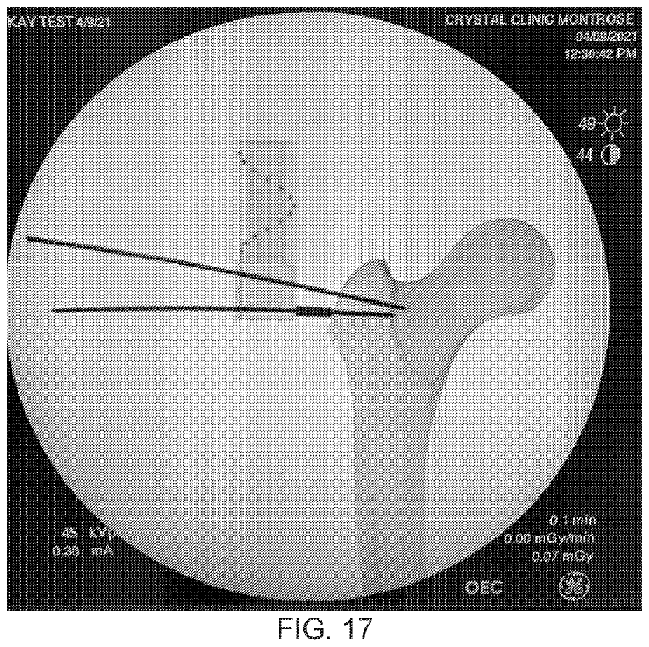

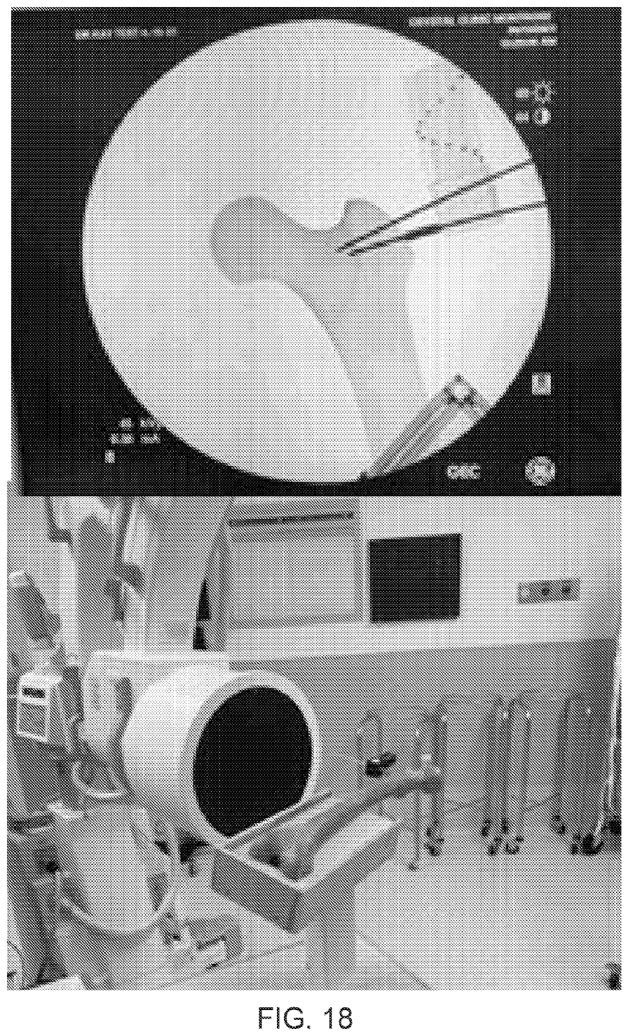

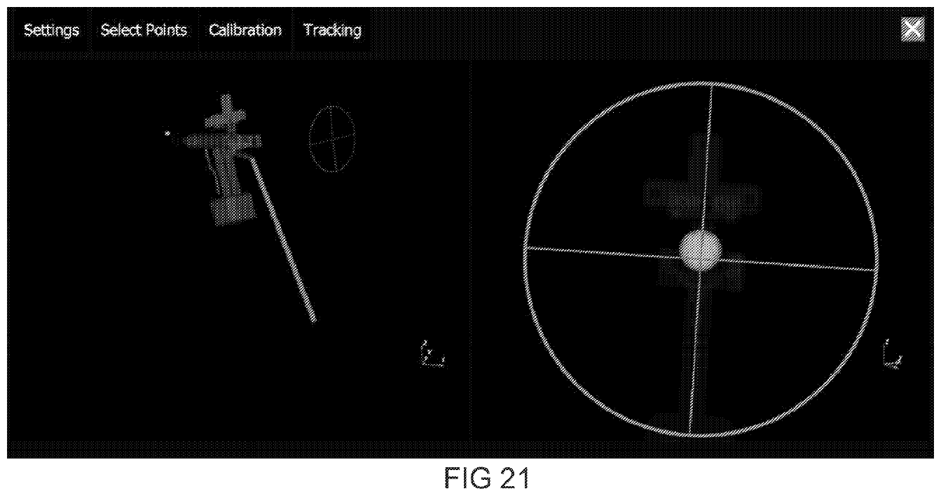

FIG. 17 illustrates the fiducial phantom 1399 with the radio-opaque fiducial balls 1420 in a regularly spaced spiral array 1422. The phantom 1399 is mounted on the guide wires pins 1420, 1422, implanted in the lateral side of a proximal femur 1450 and at least one of the pins, 1422, includes a spacer member 1425. FIGS. 18-20 illustrate how the spiral arrangement of the fiducials in the phantom show up in 2 differing 2D fluoroscopic images, and how these are marked to create the 3D coordinate reference frame that forms part of the 3D coordinate system through which the instrument travels and in which the tracking component of the present system tracks the movement of the instrument along a work-path from one point of interest to a second point of interest. FIG. 21 illustrates the GUI which the user follows.

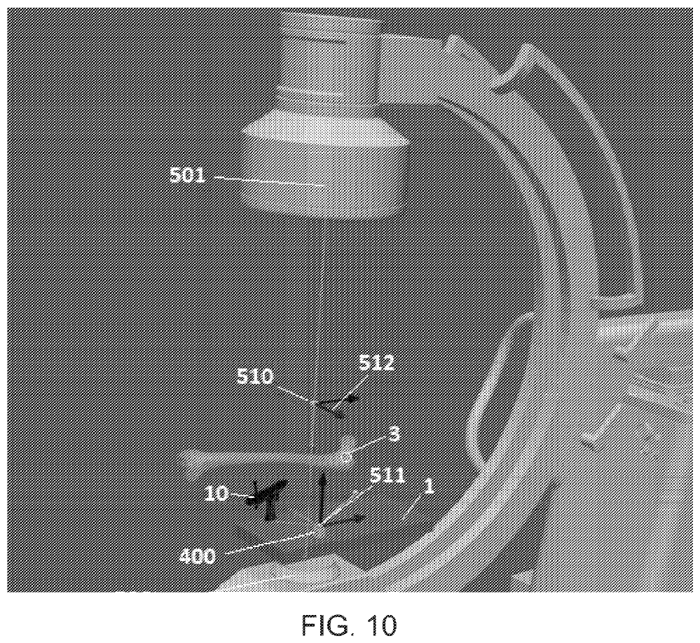

FIG. 10 shows an X-ray C-arm system comprised of an X-ray source 501 and X-ray scintillation detector plate 506 with an iso-center 510 and global frame of reference 512, with a 3-dimensional fiducial point-cloud cube 400 with local frame of reference 511. The drill handle 10, target point 3, and reference frame mechanical ground base 1 are also show. As previously stated, the target 3, and the 3-dimensional fiducial 400 must be within the field of view of the X-ray beam path and the scintillation detector screen 506. In order to locate the target 3 which has been selected in the X-ray images, we locate the fiducial 400, which is attached rigidly to the reference frame 1, which is also attached the gimbal 40 (not shown) of the present system as shown in FIG. 1 but omitted here for visual clarity, and whose position is known in the local coordinate frame 511 of the gimbal 40, in the global coordinate frame of the C-arm 512. The coordinate transform we are looking to calculate is the gimbal C arm.

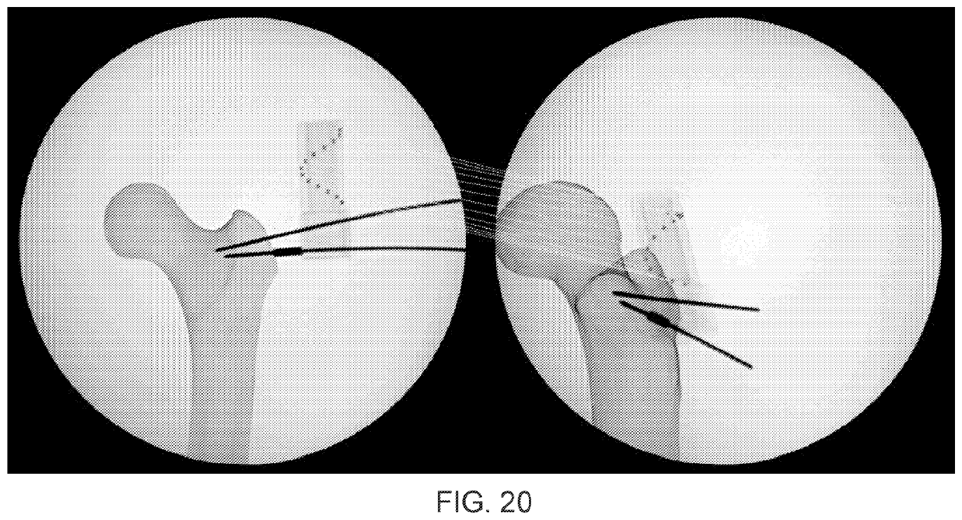

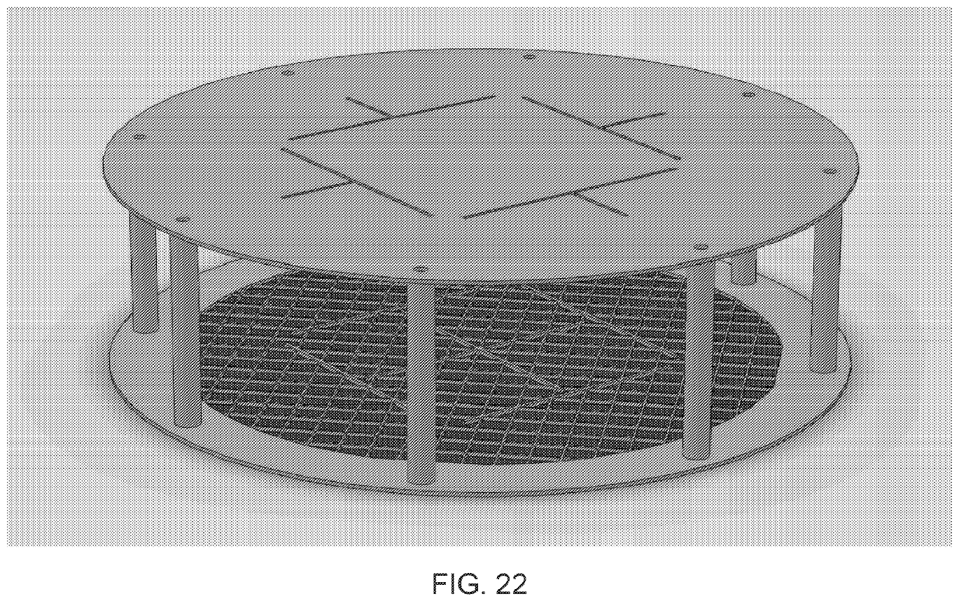

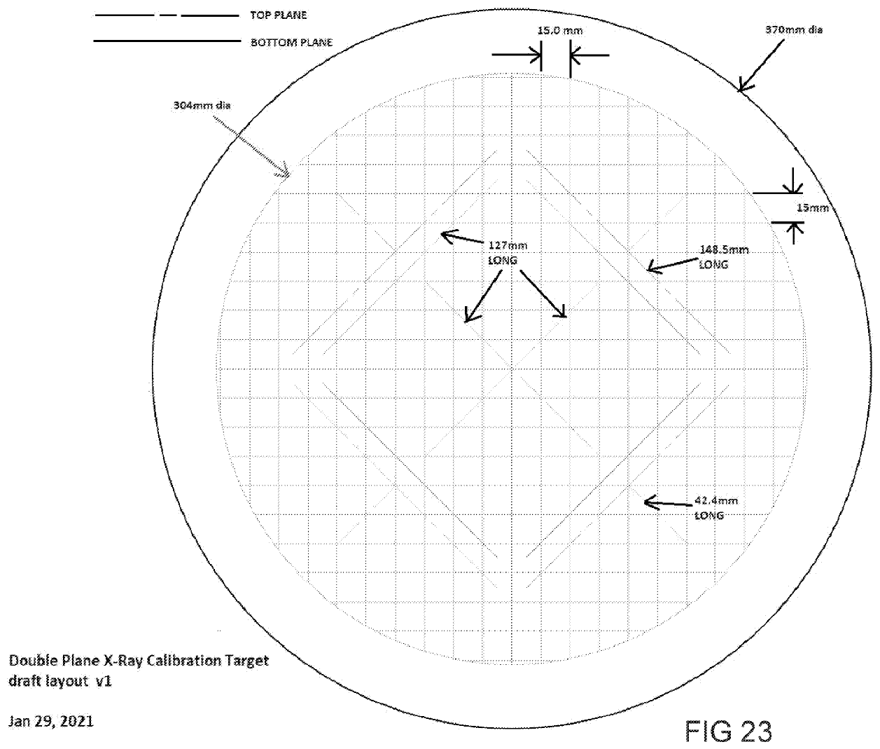

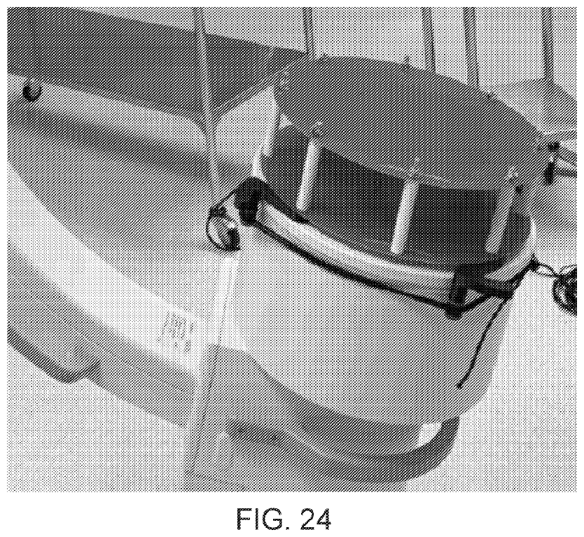

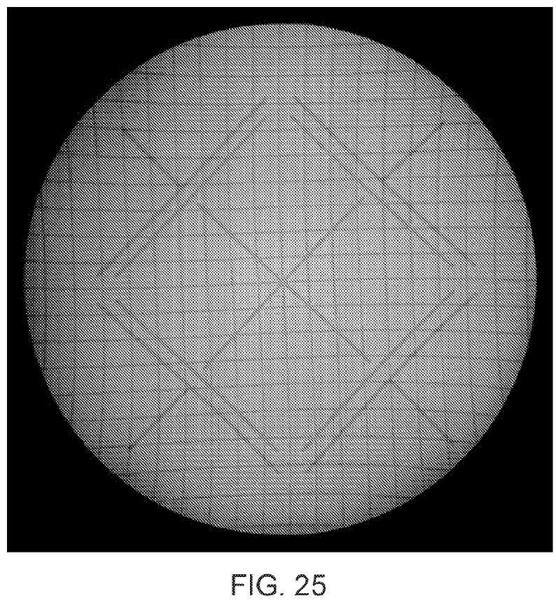

FIGS. 21-25 illustrate a calibration target 1500 in accordance with a further aspect of the present system which can be used for calibration and to compensate for distortion in the fluoroscopic images generated by the device. This target 1500 includes two parallel planes of radio translucent geometric shapes 1501 of known dimension and shape which are integral to first and second planar members 1503, 1505. The target 1500 is dimensioned to fit on the focal surface of a C-arm 1507 as is shown in FIG. 20. The radio translucent geometric shapes 1501 are advantageously formed as cut-outs in the planar members 1503, 1505. Flere, the top or outer relative to the C-arm planar member 1503 includes a cut-out which comprises a set of four lines 1510 that define a square 1511 without corners and at the mid-point of each line and of the square, the geometric shape further includes a line 1513 extending orthogonal away from the line. The bottom planar member 1505 includes a complementary geometric shape 1521, in this case, also a square, formed of four side lines 1520 having four internal lines 1523 which dissect the internal square into four equal squares and which align with the outwardly extending lines 1513 of the upper planar member. The lower planar member includes a grid of interesting ribs 1517 with interstitial square openings 1518. The lower grid includes an integral annular flange 1525 that frames the lower planar member and which includes holes that allow for radially spaced supports 1528 to hold the upper and lower planar members 1503, 1505 at a known parallel spaced relationship. An x-ray taken of this calibration target allows the software of the present system to calibrate the C-arm images so that the software can more accurately locate the tracking system on the instrument within the reference coordinate plane of the present system. FIG. 25 shows the fluoroscopic images generated by images taken with the target in the position shown in FIG. 24. The x-ray shows two images superimposed from one set of x-ray projections. The grid is on the bottom plane member and the square pattern is on the top plane member. In the x-ray, the change in the size of the square pattern shows the magnification and angle of the x-ray beam, while the grid shows the distortion.

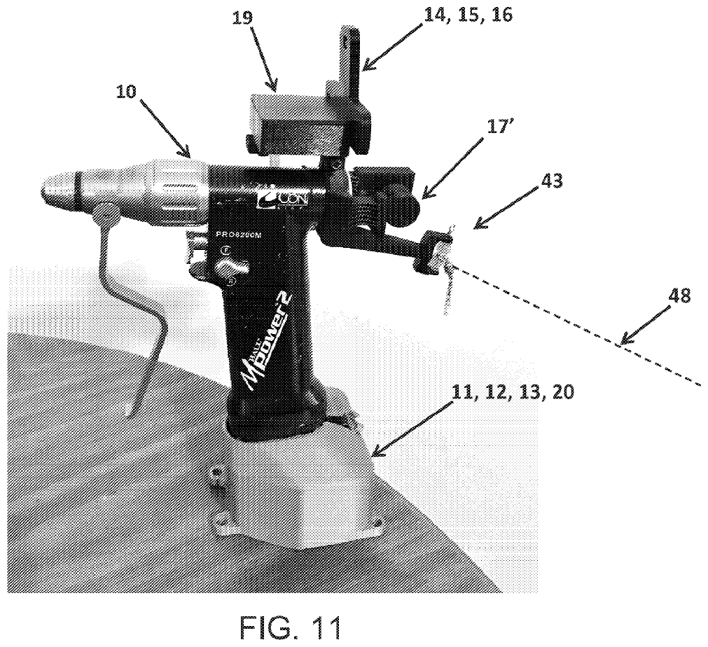

FIG. 11 shows a first embodiment with more compact housings mounted to the tool driver 10, for the various electronic components: MCU 11, Radio Tx/Rx 12/13, MUX 19, and battery housing 20; OTOF sensors 14, 15, 16; and a drill bit position sensor 17′ with a low friction yoke-mounted swivel-based ball joint attachment 43, for the draw wire 48.

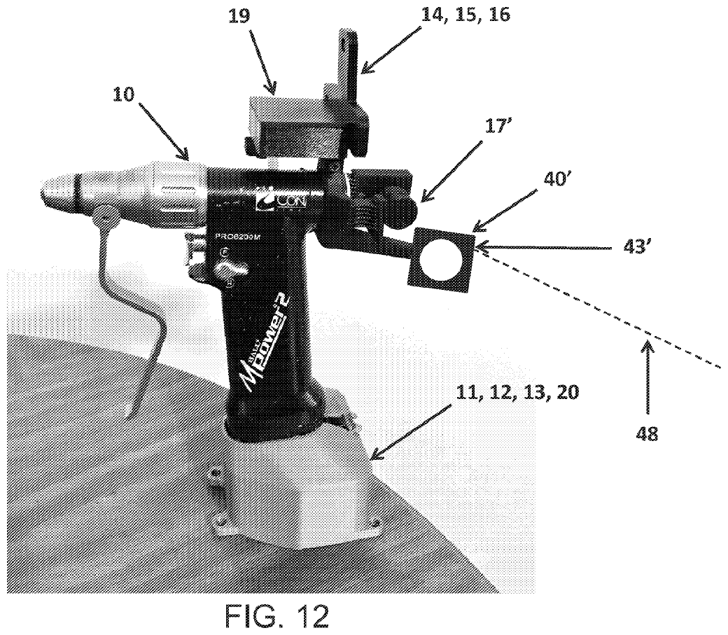

FIG. 12 shows a drawing of an alternate embodiment that has the draw wire encoder 40′ located on the tool handle instead of the gimbal 41 (not shown). By placing the draw wire encoder 40′ on the tool driver 10, and making the draw wire 48 travel through a small aperture that acts as the new pivot point 43′ (formerly the ball joint swivel 43). This has the advantage of reducing the mass of the moving parts of the gimbal 41 to permit more accurate angular tracking of the gimbal attitude. It also has the further benefit of placing as many of the sensors on the tool handle, thus permitting a high degree of integration and consolidation of the various sub systems onto one sensor system for ease of retro-fitting-an existing tool driver 10, such as by clamping the single sensor system unto the tool driver.

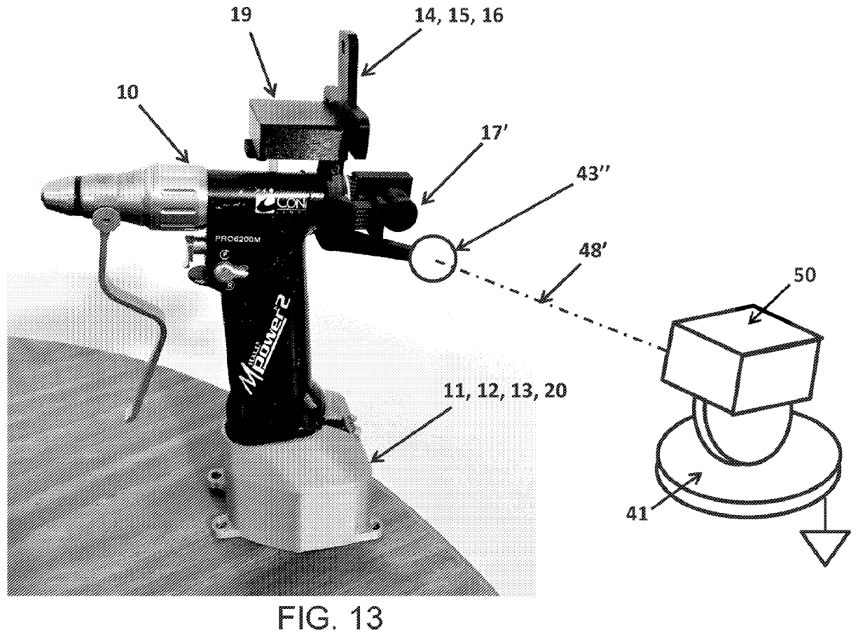

FIG. 13 shows a drawing of yet another alternate embodiment whereby the physical draw wire 48 is replaced with a wireless optical tracking and LIDAR system 50 which provides the angular attitude of the gimbal 41 via an active target tracking system focused on an optical tracking target sphere 43″ which is tracked along an axis 48′, formerly provided by a physical draw wire 48. This system has the advantage of being very unobtrusive as the operator is not encumbered by the physical draw wire 48. The distance from the gimbal 41 to the pivot point located at the tracking sphere 43″ is provided by highly accurate optical LIDAR sensor 50 mounted on the gimbal 41.

Note that there is an assumption that the system has been calibrated so that the intrinsic parameters (pixel spacing of the detector, the distance between the X-ray source and detector plane, location of the iso-center of the C-arm) are accurate and extrinsic parameters can be measured with suitable accuracy. To locate a point one needs the intrinsic and extrinsic C-arm camera parameters. As given in (Brost, et al., 2009), the camera model can be taken to be a Pinhole Camera model, with a projection matrix given by:

$$P = K [ R \vert P = K [ R \vert \qquad ( 1 )$$

The intrinsic parameters K of the X-ray “camera” can be evaluated as:

$$K = ⌈ ⌊ \vert \vert \vert SIDpSIDpoxoy 1 ⌉ ⌋ \vert \vert \vert \qquad ( 2 )$$

where
   SID is source to image detector distance.
   p is the distance between pixels in mm
   oₓ is the x location of the projected iso-center in pixels on the image.
   o_{y} is the y location of the projected iso-center in pixels on the image

The Extrinsic Parameters are given by the two rotations

RαRα
and

RβRβ
and a translation t, where t is the translation from the X-ray source to the iso-center of the C-arm. Note that in (Brost, et al., 2009) the rotation matrix given as

RαRα
is clockwise positive about the Z axis, and

RβRβ
is clockwise positive about the x-axis. In addition, the axes are aligned with the DICOM patient axes (LPS, X goes from Patient right to patient left, Y goes from patient Anterior to Posterior, and Z goes from Patient Anterior to Superior.).

The rotations are combined into a matrix R given by:

$$R = R α \cdot R β R = R α \cdot R β \qquad ( 3 )$$

From equation 1, we can project a global point

\tilde{w}∈R3\tilde{w}∈R3
to

\tilde{v}∈R3\tilde{v}∈R3
where v is a homogenous point in 2d space and w is a homogenous point with 1 as the fourth component. The projection can be written as:

$$\tilde{v} α , β = K ( R \cdot w + t ) \tilde{v} α , β = K ( R \cdot w + t ) \qquad ( 4 )$$

To solve for the global C-arm points:

$$\tilde{w} = R - 1 \cdot ( K - 1 \cdot \tilde{v} α , β - T ) \tilde{w} = R - 1 \cdot ( K - 1 \cdot \tilde{v} α , β - t ) \qquad ( 5 )$$

The 3-dimensional fiducial 400 in C-arm global coordinates 512 can be used to find the translation vector needed to translate the target 3 position into the gimbal 40 frame of reference 511. This is comprised of a translation followed by a rotation to bring the C-arm basis vectors 512 aligned with the gimbal 40 frame of reference basis vectors 511. In this way, multiple angle (>2) projections of the 3-dimensional fiducial are not needed to register the two frames of reference together, as when performing the registration using a multi-angle computed tomographic (CT) reconstruction technique.

In addition to the aspects of the disclosure previously discussed, the present system also provides for calculation and compensation of the distortion of the imaging system. This is provided by an array of fiducials of a precisely known geometric configuration set into a distortion base member. Images are taken with the distortion block in position within the focal plane of the imaging system, and measurements are taken to calculate the distortion as the image travels out from the focal point. This procedure can be undertaken on a regular basis, or as needed, for example, when the fluoroscope has been moved.

The system of the present disclosure can be characterized as incorporating various component parts, which interact in a coordinated way to function together to allow the present system to work:
1) A registration system in which a radio-translucent phantom having radio-opaque markers at a known geometric arrangement and distance are mounted from the patient as near as possible to a specific point of interest, and where the markers are viewed and marked in two 2D fluoroscopic images at planes differing from 40° to 90° to each other (such as x-rays taken using a C-arm), wo as to coordinate the marker views and enable the system to create a 3D coordinate reference system;
2) A Tracking system with sensors to track the location of a tool which holds the tracking system in the 3D coordinate reference frame in real time as it moves in 3D within the 3D coordinate reference frame;
3) A Data Input and Memory for expert based input of one or more points of interest based on expert interpretation of the 2D fluoroscopic images into the 3D reference frame to mark and remember the location of the point of interest in the 3D reference frame;
4) A Mapping System which determines a trajectory based on the point of interest in the 3D coordinate reference frame and which monitors the progress of one of more of a feature of the tool, including a tool tip, or a vector force imparted by the tool; and
5) A User Interface to give feedback to a user as to the location, attitude or progress of the tool, the tool tip, or the vector force imparted by the tool in the body.

In accordance with an additional aspect of the disclosure, a surgical procedure of method of surgery is provided which uses the targeting system of the present system in an optimal surgical workflow which allows for 3D planning intraoperatively that is patient specific, in that it is based upon actual 2D images of the specific patient and not on a generalize anatomical representation relative to palpated bony landmarks. The procedure further eliminates the need for 3D imaging, such as MRI or CT scans, which are typically performed pre-operatively.

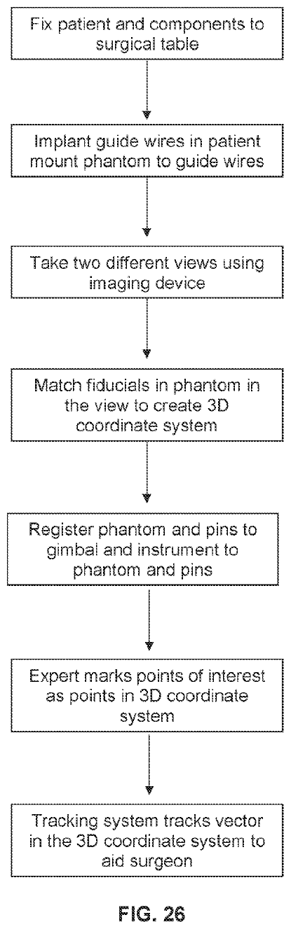

The procedure of the system is illustrated in a schematic shown in FIG. 26. The steps follow:
1) The patient and those components of this system (i.e., the gimbal and the pin driver holster) that are so secured are fixed to the surgical table.
2) A registration pin (or two pins can be placed at differing angles to the bone) is implanted freehand into the bone of interest and the registration phantom comprising the fiducial block, i.e., a radio-translucent block with embedded radio-opaque markers, is mounted from the pins to connect the phantom to a bone of interest as close as conveniently possible to the start and end point or other defined loci. Thus, the phantom and bone will move as a single solid body. The phantom contains opaque objects of known relative spacing, for example a spiral, which can be observed in the subsequent imaging. Advantageously, the block is a compact shape, such as a cylinder, which is mounted to the bone, such as by securing it to guide wires which have been implanted in a freehand surgery into the anatomical site. It is customary in many procedures to use such guide wires, so this would not necessarily entail an additional insertion procedure.
3) At least 2 different views of the registration phantom and the bone of interest, preferably including 3 AP and 1-3 lateral views, are obtained by imagining, most likely by 2D fluoroscopy, where the views are in differing planes.
4) The observed locations of the opaque objects within the registration phantom are matched, for example by a point by point click, in the 2D fluoroscopy views, such as by measuring, and comparing a sufficient number, i.e., some or all, of the fiducials to their known spacing, in order to create a virtual 3D coordinate system from the 2D images and to compensate or correct for fluoroscopy distortion.
5) The phantom and the pin is registered to the gimbal to place the bone, gimbal, and the pin driver is registered by touching and marking, such as by clicking registration points on the registration pins or pins, and the phantom to register it in the virtual 3D coordinate system. Even though the phantom is fixed to the bone, this registration step is required because the registration pins are placed by freehand.
6) Expert observation (for example, by the surgeon or a trained assistant) of the 2D images, and points of interest in the differing 2D views are noted and marked, such as by identifying these locations within the 3D coordinate references system or the 2D views used to create this system, and using the same calculations that were used to create the 3D coordinate system, the points of interest are brought or marked within into the 3D coordinate system. This aspect of the system allows an expert to observe the images pre-operatively or even intraoperatively as an initial step in the surgery and create a 3D surgical plan by placing points of interest onto the 2D images. The software of the present system thus, enters those points into the 3D coordinate reference system. Thus, in contrast with the prior art surgical workflow in which the surgeon places points of interest onto the fluoroscopy images of the patient anatomy, the present system introduces the patient's anatomy into the 3D surgical plan through the surgeon's observation of the fluoroscopy images. Prior art planning systems start by creating a 3D model of the patient's anatomy, then create a 3D surgical plan by applying some protocol to that 3D model of the patient's anatomy. It can be difficult/impossible to create an accurate 3D model of the patient's anatomy from 2D images. For many procedures, a full 3D model of the patient's anatomy; it not necessary, it is better to know the location of specific points of interest.
7) The surgery is performed according to the 3D surgical plan beginning by placing the instrument tool tip at the entry or beginning point of the work-path and as guided by the instrument system of the present system including the location definition portion and the tracking component which determines the alignment of a vector relative to the bone within the reference frame. If the bone moves, then it may be desirable to re-register the bone into the virtual 3D coordinate system without requiring additional fluoroscopy images. If the bone is in a part of the anatomy that is subject to several joints, or which is difficult to secure, an inertial unit can be placed on the registration pin to track the bone movement in the virtual 3D coordinate system.

In accordance with the patent statutes, the best mode and preferred embodiment have been set forth; the scope of the invention is not limited thereto, but rather by the scope of the attached claims.

1. A positional determination and guidance system to guide the user of a surgical tool having a terminal workpiece along a work path in a patient's body from an initial start point to a determined end point, comprising:
a base member at a fixed reference point which supports a two-member virtual robotic arm system comprising a variable-length first arm which is comprised of a virtual link which extends at an angle a and can be extended by a value n from a first length to a second length, and which comprises a length sensor capable of determining the value n, and at least one angular sensor capable of determining angle a,
a hand-held tool serving as a distal link in the form of a second fixed-length arm which is linked to the first arm, and which has a plurality of angular sensors and a plurality of time of flight transceivers for determining absolute distance relative to a reference plane,
a CPU having memory and machine readable code to determine the progress of the terminal tool in 3D along the work path from the initial start point toward the determined end point,
means for a user to choose a point of interest and to log it in 2D in the memory of the CPU and to thereby determine the work path, and
a display that informs the user as to the guidance of the terminal workpiece along the work path.

2. A positional determination and guidance system as set forth in claim 1, that uses a combination of radio frequency wireless communication and electrical communication to form the virtual link between the hand-held tool and the base member.

3. A positional determination and guidance system as set forth in claim 2, further including time of flight transceivers, means to create a reference plane and to calculate a time of flight determination between the time of flight transceivers and the reference plane.

4. A positional determination and guidance system as set forth in claim 1, wherein the time of flight transceivers generate light pulses in the visible or near infrared spectrum for use in the guidance of the terminal workpiece.

5. A positional determination and guidance system as set forth in claim 3, wherein the positional information is provided through the use of angular measurements provided by MEMS IMU's used in conjunction with measured and known distances via the forward kinematics equations to determine the position and trajectory as a function of time.

6. A positional determination and guidance system as set forth in claim 3, wherein the combination of the forward kinematically derived positions and angles are compared to absolute distances and angles as determined by the time of flight transceivers mounted on the distal link.

7. A positional determination and guidance system as set forth in claim 3, wherein the forward kinematic equations in combination with the time of flight information is used by the CPU to perform calculations to derive the positional information and angular approach.

8. A positional determination and guidance system as set forth in claim 3, wherein the tool handle comprises the distal link.

9. A positional determination and guidance system as set forth in claim 2, wherein the time of flight transceiver is an optical time of flight transceiver.

10. A positional determination and guidance system as set forth in claim 7, further including a spiral fiducial phantom mounted to guide wires implanted into bone which are used to determine a 3D reference coordinate system for the system based upon 2D fluoroscopic images.

11. A positional determination and guidance system for a surgical tool path determination as described in claim 1, further including video cameras to create video images and where the video images are also used simultaneously in the guidance of the terminal workpiece along the work path.

12. A positional determination and guidance system as set forth in claim 1, including the further use of 2D fluoroscopic imaging to derive the tool work path determined end point.

13. A positional determination and guidance system as set forth in claim 1, wherein the length sensor is an optical sensor.

14. A positional determination and guidance system as set forth in claim 1, wherein the accuracy of the guidance of the work path to the determined end point is at least +/−2 degrees.

15. A surgical instrument system which is used by a user during a procedure holding an instrument having a tool to guide the tool along a work path from an initial starting point to a desired end point in a relevant portion of a patient body in a working field comprising:
a registration pin which is implanted in the working field such that it moves with the relevant portion of the patient body,
a plurality of radio-opaque fiducials held in a spiral of a known geometric relationship within a radio-translucent block to form a phantom and the phantom mounted to the registration pin so as to enable the surgical instrument system to develop a 3D coordinate framework and means to define locations on the work path with the coordinate framework,
machine vision software loaded in a machine which interprets the locations on the work path and displays them on a monitor, and means for the user to define on the monitor in two planes a location relative to the patient body for at least two of the fiducials and the initial start point which location is determined by 2D fluoroscopy and which initial start point is determined by a human expert as part of a pre-operative or intra-operative planning procedure,
where the instrument system includes the means for the human expert to register the initial start point to the surgical site within the coordinate framework, and
an instrument having a tool and which carries sensors to determine the position of the workpiece relative to the 3D coordinate framework,
wherein the system determines and progressively displays in real time, in two dimensions for the initial start point and a determined end point, a work path from the initial start point to the determined end point in the coordinate framework so as to guide a user holding the surgical instrument in forming the work path from the initial starting point to the determined end point.

16. A method of planning of a medical procedure on a body comprises the steps of:
implanting a registration pin in the body;
connecting a registration phantom comprising a radio-translucent fiducial block holding a series of radio-opaque fiducials in a known geometric relationship to the registration pin;
taking at least 2 different 2D fluoroscopic images in different planes of the registration phantom;
comparing the locations of the fiducials in the 2D views to the known geometric relationship;
creating a 3D coordinate system around the registration phantom; observing the 2D fluoroscopic images and marking at least one point of interest and coordinating the location of the point of interest in the 3D coordinate system; and
performing the medical procedure using an instrument having a tracking location which tracks the location of the point of interest within the 3D reference frame.

17. (canceled)

18. (canceled)

19. (canceled)

20. (canceled)
